(19) United States
(12) Patent Application Publication
(10) Pub. No.: US 2020/0405232 A1
(43) Pub. Date: Dec. 31, 2020

(54) FLEXIBLE CIRCUIT FOR DETECTING LIQUID PRESENCE

(71) Applicant: Digital & Future Technologies Limited, Colchester (GB)

(72) Inventor: Christopher John Crockford, Colchester (GB)

(21) Appl. No.: 16/970,244

(22) PCT Filed: Feb. 15, 2019
(86) PCT No.: PCT/GB2019/050411

§ 371 Date: Aug. 14, 2020

(30) Foreign Application Priority Data
Feb. 15, 2018 (GB) 1802496.8

(51) Int. Cl. A61B 5/00; A61B 5/0205; A61B 5/04; G01N 27/22; G01L 5/1627

(52) U.S. Cl. CPC A61B 5/6801; A61B 5/0205; A61B 5/7445; A61B 5/7405; A61B 5/7455; A61B 5/6807; A61B 5/0816; A61B 5/04012; G01N 27/223; G01N 27/228; G01L 5/1627; A61B 2562/0261; A61B 2562/164; A61B 5/6831

(57) ABSTRACT

A flexible circuit comprising two or more circuit layers separated by one or more insulating layers; a plurality of nodes located on each of the circuit layers; and a processor configured to estimate the distribution of liquid within the circuit from the capacitance measured at one or more of the nodes.

### FIELD

This invention relates to a circuit for calculating the three-dimensional distribution of liquid within the circuit and force acting on the circuit.

### BACKGROUND

It is often desirable for physical properties to be measured on or around the human body; the fields of medicine and personal fitness are two areas where this is particularly common. Conventional technologies often rely on bulky or intrusive apparatus that can impact the movement or comfort of the individual. Recent improvements in the field of flexible electronics are allowing for devices that are sufficiently flexible, lightweight and durable to enable electronics to be incorporated into clothing. Such flexible electronics may also provide benefits in the field of medicine where they may be incorporated into clothing, wound dressings, bandages, support bandanges and compression socks.

There is a need for an improved mechanism for measuring the physical state of a flexible circuit.

### SUMMARY OF THE INVENTION

According to the present invention there is provided a flexible circuit comprising two or more circuit layers separated by one or more insulating layers, one or more nodes located on each of the circuit layers, and a processor configured to estimate the distribution of liquid within the circuit from at least one of the electrical conductance and the capacitance measured at one or more of the nodes.

The processor may be further configured to, by comparing one or more of the electrical conductance and the capacitance with known values, at least one of: identify the liquid, determine the pH of the liquid, and determine the salinity of the liquid.

The processor may be further configured to estimate the rate at which liquid passes through the circuit by comparing the distribution of liquid within the circuit two or more points in time.

The processor may be further configured to estimate the distortion of the circuit from the capacitance measured at one or more of the nodes.

One or more of the circuit layers may further comprise a strain gauge.

The processor may be further configured to increment a counter when the distortion exceeds a predetermined threshold.

The processor may be further configured to continuously record the distortion of the circuit.

The processor may be further configured to compensate for certain predefined distortions by ignoring the distortion detected by a subset of the nodes.

The circuit may be arranged such that, when affixed to a user and in use, the circuit layers are situated for detecting distortion caused by at least one of: cardiovascular activity, and respiration.

The processor may be further configured to estimate the heart rate or the respiratory rate of a user based, at least in part, on the detected distortion caused by cardiovascular activity or respiration respectively.

The processor may be further configured to receive data from another flexible circuit and, in dependence on the received data and the detected distortion caused by at least one of cardiovascular activity and respiration, to determine a pulse wave velocity.

The one or more of the nodes may be arranged such that, when the circuit is affixed to a user and in use, the nodes measure the electrical activity of the skin of the user underlying the circuit.

The flexible circuit may further comprise a common ground wire for connecting to another flexible circuit and wherein the processor is further configured to receive data from two connected flexible circuits and, in dependence the received data and the detected electrical activity of the skin of the user underlying the circuit, to determine an ECG reading using Einthoven's triangle effects.

The processor may be further configured to estimate the heart rate of a user based, at least in part, on the detected electrical activity of the skin of the user underlying the circuit.

One or more of the circuit layers may be configured to generate heat by passing a current through a resistive element.

One or more of the nodes may be arranged such that, when the circuit is affixed to a user and in use, the nodes apply an electric current to the skin of the user underlying the circuit.

The flexible circuit may further comprise at least one of: a gyroscope, and an accelerometer, configured to output movement signals to the processor, wherein the processor is configured to use the movement signals to calculate the position of the circuit.

The processor may be further configured to determine the energy expended in movement in dependence on changes in the position of the circuit and the mass of any object affixed to the circuit.

The flexible circuit may further comprise a GPS tracking system configured to determine the location of the circuit.

The processor may be further configured to receive a location signal from an external device and use the location signal to determine the location of the circuit.

One or more of the nodes may comprise an LED and a light sensor and are arranged such that, when the circuit is affixed to a surface and in use, these nodes measure the reflectance of the surface.

Two or more of the nodes may comprise an LED and a light sensor and are arranged such that, when the circuit is affixed to a surface and in use, the reflectance of the surface under these nodes is compared.

One or more of the nodes may comprise a vibration-sensitive piezoelectric element and wherein the processor is configured to record the vibrations detected by the piezoelectric elements.

Two or more nodes may comprise vibration-sensitive piezoelectric elements and are arranged such that, when the circuit is in use, the processor is configured to estimate the source of vibrations by comparing the vibrations detected by the piezoelectric elements.

The flexible circuit may further comprise one or more piezoelectric elements arranged such that, when the circuit is affixed to a surface and in use, the piezoelectric elements exert pressure on an affixed surface when stimulated by an applied electric field.

The nodes may be dimensioned so as to allow the reception and transmission of radio frequencies.

The flexible circuit may further comprise a communications interface configured to transmit or receive data to/from one or more external devices using at least one of: a serial connection, a parallel connection, a USB connection, an ethernet connection, a flexible flat cable connection, radio, Bluetooth, WiFi, and Near-field Communication.

Data collected by the flexible circuit may be uploaded to a distributed ledger.

The energy of received radio waves may be used to power the circuit.

The processor may be further configured to enter a low power state in which only data critical to the operation of the circuit is transmitted when the circuit is first powered on or in response to an input from a user, and enter a high power state, in which all data may be transmitted, when the circuit has been powered on for a predetermined length of time or in response to an input from a user.

The flexible circuit may be further configured to not take measurements at the nodes when in the low power state.

The flexible circuit may further comprise a proximity sensor and wherein the processor is configured to enter a high power mode in response to the proximity sensor detecting close proximity of a surface.

The flexible circuit may further comprise an inductor arranged to passively harvest energy from ambient magnetic fields as the flexible circuit moves therethrough.

The flexible circuit may further comprise an indicator configured to provide an indication of measured parameters to a user, the indicator comprising at least one of: a video display, an e-ink display, blinking light(s), LED(s), a vibration mechanism, a speaker.

The indicator may be configured to provide an indication to a user in response to expiration of a predetermined period of time.

The flexible circuit may further comprise a capsule for storing a medicament, configured to release the medicament in response to a detected change in the pH of the liquid within the circuit.

The flexible circuit may further comprise a capsule for storing a medicament, configured to release the medicament in response to a predetermined applied distortion.

The capsule may be configured to be soluble.

The capsule may be configured to fracture upon receiving the predetermined applied distortion.

The capsule may comprise a frangible region inclined to break upon receiving the predetermined applied distortion.

A medicament may be held in the one or more insulating layers.

The flexible circuit may further comprise an LED configured to output light of a predetermined wavelength onto the medicament.

The flexible circuit may further comprise one or more sheets of graphene for filtering out light of a predetermined wavelength.

The processor may be further configured to produce a three dimensional image of a parameter measured at the nodes.

The flexible circuit may further comprise an RFID tag storing at least one of: a unique identification number, the time or date when the circuit was installed, the operational performance of the circuit, the status of an object affixed to the circuit.

The flexible circuit may further comprise a timing mechanism for providing a clock signal to the processor and wherein the processor is configured to record the time of the occurrence of one or more: a measurement taken by the circuit, a signal received by the circuit.

The processor may be further configured to determine that a packaging has been tampered with if the distortion measure at one or more of the nodes exceeds a predetermined tamper threshold value.

The processor may be configured to communicate with a network of flexible circuits.

Each circuit layer may be connected to a respective processor and wherein measurements taken by the nodes on each circuit layer are relayed to their respective processors.

The flexible circuit may be incorporated into at least one of: a wound dressing, a bandage, a support bandage, compression socks, and clothing.

The circuit layers may comprise printed graphene circuitry.

An insulating layer may be imbued with an electrolyte and two circuit layers adjacent to the imbued insulating layer comprise electrodes, wherein the imbued insulating layer and the electrodes operate as an electrochemical cell.

The flexible circuit may further comprise one or more power harvesting piezoelectric elements configured power the circuit using the harvested from distortions of the circuit.

The flexible circuit may further comprise one or more magnetic elements for affixing the circuit to a surface.

The flexible circuit may further comprise a barcode encoded with circuit identification information.

### DESCRIPTION OF THE DRAWINGS

The present invention will now be described by way of example with reference to the accompanying drawings. In the drawings:

FIG. 1A shows a schematic diagram of an example circuit arrangement.

FIG. 1B shows a schematic diagram of an example circuit comprising several stacked circuit layers.

FIG. 2 shows a schematic diagram of two adjacent circuit layers and their respective nodes.

FIG. 3 shows an exemplary use of a system as part of a wound dressing.

FIG. 4 shows a top-down schematic diagram of a single circuit layer.

### DETAILED DESCRIPTION

The following description is presented to enable any person skilled in the art to make and use the invention and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown but is to be accorded the widest scope consistent with the principles and features disclosed herein.

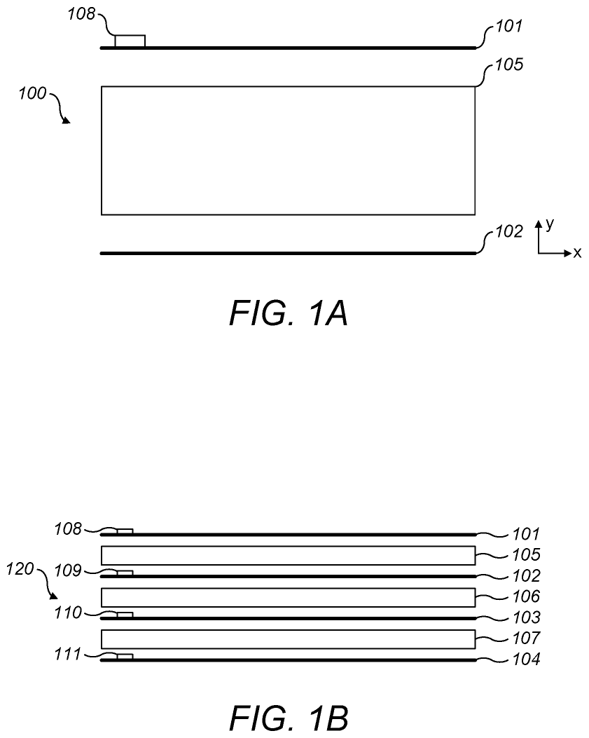

FIG. 1A is a schematic diagram of a flexible circuit 100, comprising two circuit layers 101, 102 separated by a layer of insulating material 105. The circuit may be printed onto a substrate. The substrate may be the insulating layer itself, or a distinct material like a thin flexible insulating solid or fabric. As shown in FIG. 1B, the basic circuit structure illustrated in FIG. 1A can be repeated by stacking circuit layers 101, 102, 103, 104 separated by insulating layers 105, 106, 107. In this structure the insulating layers alternate with the circuit layers through the thickness of the circuit (in the y-direction as shown). It is not necessary for the circuit layers to be equally spaced as they are shown in FIGS. 1A-b; depending on the application it can be advantageous to have different distances between the layers. It is possible to stack any amount of circuit and insulating layers, provided there are at least two circuit layers and one insulating layer between them.

A processor 108 is used to receive data collected on one or more of the circuit layers. Each layer may have an independent respective processor 108, 109, 110, 111, as shown in FIG. 1B, or a single processor may be used to centrally collate the data from several circuit layers. In one implementation, multiple slave processors may operate under the control of a single master processor. Multiple circuits may communicate in a master-slave arrangement to form a network of flexible circuits. The processor may be a simple logic unit or may include other common computer architecture such as control units, memory units, a communications interface (inputs and outputs). These inputs and outputs could be conventional communication ports such as serial ports, parallel ports, USB ports, ethernet ports, though given the advantageously thin and discreet nature of the circuitry, the inputs and outputs should be similarly thin and discreet, for example, one or more flat flexible cables (FFCs) could be used.

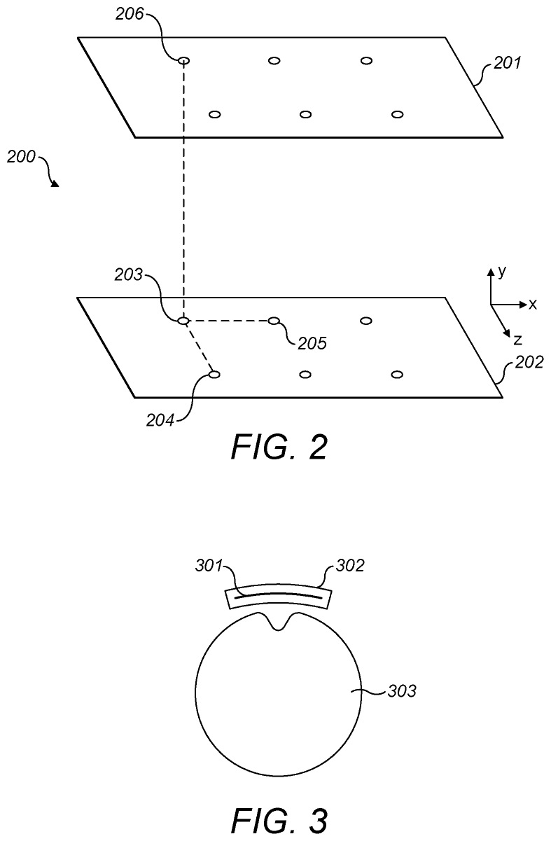

FIG. 2 shows an expanded view of two adjacent circuit layers 200. The substrate between the circuit layers has been omitted from this figure for clarity. Measurements are taken by the circuit at locations on the circuit layer(s), referred to as nodes. Each circuit layer 201, 202 shown includes a plurality of these nodes 203, 204, 205, 206. The nodes may be spatially distributed across the circuit layer, for example in a regular or irregular arrangement. The measurements taken by the nodes may include one or more of: voltage, current, electrical resistance/conductance, electrical reactance/susceptance, electrical charge, electrical power, inductance, capacitance, electrical impedance, magnetic flux and magnetic field. Various examples of the measurements that may be taken at the nodes and the specific apparatus necessary are detailed in the paragraphs below. These nodes are preferably arranged in a lattice for ease of use and manufacture but could be placed arbitrarily. The lattice arrangement conveniently allows the circuit layers to be manufactured in large sheets and be cut to the required size by a user (including a personal user or a medical professional applying the circuit to a patient). Where the circuit is to be cut to size by a user, the removal of a portion of the circuit layer (and the nodes therein) should not affect the functioning of the remaining measurement nodes. That is, the operation, or function, or operative state of a node may be independent of the operation, function or operative state of each other node (either on the same circuit layer or on each of the circuit layers). For example, the nodes may be either wired in parallel or each node may be connected individually to the processor. In other words, each node may be electrically routed to the processor independently of the other nodes.

The nodes on a single layer are described as lying on the x-z plane, with the layers being spaced apart in the y-direction, though this description is a simplification for ease of reference, particularly as the layers bend and may not remain substantially planar. The nodes may be individually addressable by the processor or may provide their readings in an aggregated form. The measurements taken at the nodes are generally strongly dependent on the current state of the insulating layers. The arrangement of the measurement nodes on multiple circuit layers, each separated by an insulating layer, forms a 3D dynamic reaction matrix. Measurements taken at the nodes allow for the coarse measurement of the properties of the flexible circuit and interpolating between the nodes allows for the estimation of the properties of the entire matrix.

From the manufacture of the circuit, the materials used, and from testing, the response of the circuit to various external stimuli can be quantified. One consideration which may be of importance is how the material used to form the insulating layer behaves under various conditions, as generally, this material will generally comprise the majority of the system in terms of volume/bulk.

Such flexible circuitry is suited for incorporation into fabrics such as regular clothing (e.g. socks, trousers, shorts, underwear, shirts, t-shirts, jumpers, hats), sporting/exercise clothing (e.g. socks, shorts, jerseys, protective pads, lame), bandages, support bandages, wound dressings and compression socks. An exemplary use for the circuit is shown in FIG. 3; here, the circuit 301 is embedded in a wound dressing 302 that is covering a wounded body part 303.

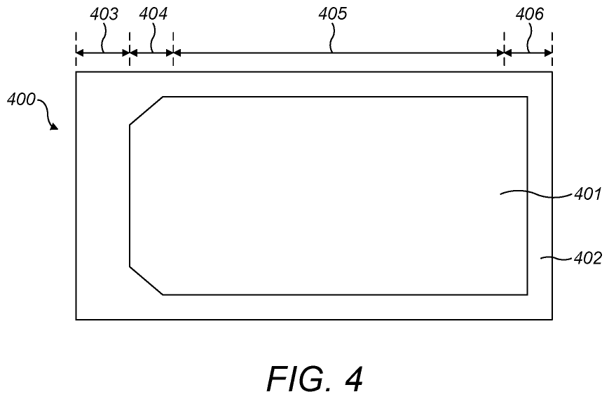

A top-down view of an exemplary circuit layer 400 is shown in FIG. 4. In this example, measuring circuitry 401 is printed onto a substrate 402. For illustrative purposes, the circuit layer is shown divided up into regions, with one of the regions being electronics carrier region 403, which in this example is shown on the far left. This region contains the processor and/or a connection to conventional electronics (for example by way of an FCC port). This region may be adjacent to a transition layer 404 which provides a link between the electronics carrier region and the measurement region 405. The measurement region is where the nodes are located. This example also includes an adhesive region 406 for fixing the circuit layer either to an insulating layer or to the object that is to be monitored using the circuit. Thus, in general, the circuit layer comprises a number of regions, which in this example include an electronics carrier region 403, a transition layer region 404, a measurement region 406 and an adhesive region 406. Though these regions are shown as contiguous, there may be significant overlap between the regions or multiple instances of a particular region, for example there may be multiple adhesive regions. The adhesive region may comprise magnets that allow the circuit to adhere to a magnetically receptive material.

Such a circuit can be used to determine the presence of liquid within the circuit. For example, a change in the capacitance across two separated conductors (e.g., within the measurement region 405) can be indicative of the presence of a liquid. As capacitance scales with the dielectric constant of the material between the conductors, this method can be used to determine fluid presence through measured changes in capacitance. The two conductors may be similarly shaped or may have different shapes. Knowledge of the specific shape of the conductors is not essential, though it may be advantageous for achieving a predictable electronic response or for ease of manufacture. Common shapes used for such applications include planar plates, parallel strips and parallel wires. The conductors may need to be insulated, perhaps by coating with an insulating layer, to avoid a current flowing from one conductor to the other which may occur when the separation is saturated with an ionic liquid.

If the conductors are not insulated, then a flowing current itself may be used to determine the presence of a liquid. The impedance between the two conductors will be reduced when a fluid is present in the separation and thus the measured impedance (or the resulting current) can be used to derive the presence of fluid.

Most liquids, including acids, bases, blood and even de-ionised water can be sensed by these methods. A binary indication of liquid presence may be given once the measured value (e.g. a change in capacitance) reaches a threshold value, or a continuous indication of liquid levels present may be given. These methods may further be used to identify the amount of liquid present, as both the capacitance and conductance will increase with increased liquid content until saturation is reached.

Additionally, the circuits can be used to identify the kind of the liquid present. Both conductance and capacitance increase as the number of free ions in a liquid increases, allowing different types of liquid to be distinguished by for instance, comparing the capacitance and conductance/impedance with known values to differentiate e.g. blood from water from salt water. This can, therefore, provide an indirect measure of properties such as pH and salinity, providing useful information as, for example, the fluids discharged by a wound are known to have a different pH depending on whether the wound is infected. Values of electrical properties (such as capacitance and conductance/impedance) for one or more different liquids may be stored in a memory unit within the processor.

The spatial distribution of liquid across a single circuit layer can be determined by taking measurements at multiple nodes, with a higher density of nodes allowing for a higher resolution liquid distribution to be determined. Incorporating measurements made by further nodes on other circuit layers, separated in the y-direction, allows a 3D distribution to be determined by the processor(s). The spatial distribution of liquid throughout the circuit allows the entry point of the liquid to be determined, for example, a wound dressing may determine whether a liquid originates from the wound or from an outside source, thus providing advanced monitoring and insight into the health of a patient. Monitoring the change in the distribution over time conveys further useful information, including how much liquid is being introduced to the circuit, which could be used to measure blood loss from a particular wound. Monitoring the liquid passthrough in a situation where liquid enters from one side of the circuit and exits from the opposite side can also provide useful information, for example in determining how much fluid is being lost through perspiration. The circuit and insulating layers may be made or cut so as to create free space through which liquids can more easily move. More or less free space will be needed depending on whether a greater or lesser liquid passthrough is desired for a particular application.

The functionality and applicability of the circuit can be further improved by configuring it to measure and/or calculate further relevant parameters. Such parameters may require additional measurement apparatus or may be derivable using the apparatus as described above.

One such relevant parameter is the distortion of the circuit. The measured distortion may include one or more specific forms of distortion such as the warping, twisting, bending, buckling, stretching, compressing, or one or more derivable parameters such as force, strain, stress, pressure, tension, torsion and weight. One method of measuring the distortion within a single circuit layer (in the x-z plane) is to incorporate a strain gauge wherein the applied force, pressure, tension, weight etc. results in a change of electrical resistance which can be measured, for example in foil strain gauges or piezoresistive elements.

A useful method of measuring the distortion between circuit layers (in the y-direction) is to monitor for changes in the capacitance between circuit layers. Capacitance generally scales as the inverse of the distance between conductive plates, and so changes in the distance between circuit layers will manifest as a change in the measured capacitance. Using this method, it is useful but not necessary to know the area of the conductive plates used for the measurement and the dielectric constant of the insulating material between the plates.

There may be a variety of applications in which determining the distortion of the circuit is desirable. For example, when the circuit is incorporated into a support bandage it may desirable to know how a patient has moved a certain limb. When the circuit is incorporated into the insole of a shoe it may be desirable to know the pressure distribution caused by the sole of a foot. When the circuit is incorporated into a sterile wound dressing it may be desirable to know whether the packaging has been tampered with (e.g. if it has been determined that the distortion has exceeded some predetermined tamper threshold value).

The processor may record a continuous variation in the distortion or it may only record once a predetermined threshold amount of distortion is measured. Once such a threshold is met, the processor may begin continuously monitoring the distortion or simply add to a counter. The threshold may be set by a user and may be constant, or such a threshold may be adaptive. The threshold may be adaptive, for example, it may be increased if the threshold level is met too frequently or for long periods of time.

The processor may compensate for distortions caused by factors that are not intended to be measured by, for example, neglecting the measurements made by a subset of the nodes (for example, those on a specified layer) or by ignoring particular distortions. Such compensations can be useful in situations where clothing is worn over a bandage or wound dressing containing the circuit and measuring distortions caused by the clothing is not desirable. Conversely, specific distortions can be targeted, measured and recorded, for example, when used in sporting equipment (e.g. rugby jerseys, fencing lame) where determining the specific location, time and force of an impact is desirable. In other words, the processor may be configured to measure a set of one or more targeted distortions, and to compensate for non-targeted distortions.

The circuit may also be configured to measure a heart rate. This can be aided by positioning the circuit on the body in a position where expansions and contractions caused by the cardiovascular activity can be measured, for example at the wrists, neck and chest. The movements of the innermost circuit layer (i.e. the closest layer to the skin) relative to one or more outer circuit layers (i.e. layers further from the skin), or the stretching in the x-z plane of the innermost circuit layer can be detected and timed providing a measure of the heart rate of a wearer. Similarly, a person's respiratory rate can be found by detecting and timing the expansions and contractions of the chest. Related methods could be used to determine the blood pressure of a wearer, with the circuit acting as a sphygmomanometer enabling oscillometric measurements to be made by monitoring the increases in pressure exerted on the innermost circuit layer.

The use of two circuits spaced apart or of one positioned over a blood vessel of sufficient length can be used to determine blood pressure by using pulse wave velocity techniques. Two spaced apart circuits can be used to establish pulse arrival times or one circuit may be used to track a pulse wave as it travels along the structure. A similar method could also be used to model the 3D path of temporarily swollen veins or to characterise localised swelling.

In another implementation, the nodes on the circuit layers may act as EEG/ECG/EOG electrodes that are in contact with or within a specified height of the skin, for example within 0.1, 0.2, 0.5, 1, 2 or 5 millimetres. As with the majority of the possible measurements described herein, the EEG/ECG/EOG measurements are able to benefit from the 3D structure of the circuit. Taking such measurements on multiple nodes on multiple layers allows a 3D image of the measured quantity to be generated. By taking ECG readings using several separated circuits connected by a common ground wire it is possible to take advantage of the Einthoven's triangle effects.

By using the circuit to generate electromagnetic fields and waves by way of electrodes, solenoids and antennae, the circuit can be used to influence the biological and rheological properties of nearby objects. It is known that various electric and magnetic fields and electromagnetic waves can affect various properties of blood. For example, when the circuit is incorporated into a wound dressing the circuit may increase blood clotting by applying a pulsed magnetic field. A related improvement can be made by arranging electrodes on the innermost circuit layer such that they apply an electric current to the skin. The application of carefully regulated electric currents to the skin can cause nearby muscles to activate, providing health and cosmetic benefits. Furthermore, electric currents can be used to relieve pain through the process of transcutaneous electric nerve stimulation (TENS). Passing a current through a resistive element on a circuit layer (without applying it to the skin) would allow the circuit to generate heat, providing warmth to the circuit and/or a wearer.

A further improvement to the described circuit could be implemented by incorporating gyroscopes and/or accelerometers into the system. These devices, separately or in conjunction with the distortion measurements described above, can be used to monitor and track the circuit's location in 3D space. Using such measurements, the processor may track the 3D movement of the circuit and it may further estimate the amount of energy that was expended in moving the circuit in such a way (e.g. using the known mass of any object affixed to the circuit). The processor may for example track the movement of the circuit over a specified time period, and calculate the corresponding energy expenditure over that time period. Such additional components may be combined with or replaced by a GPS tracking system which could be incorporated into the circuit itself or provided by a nearby external device, such as a smart phone. GPS tracking would further improve the monitoring and tracking of the circuit's location and movements, in particular when used with a timing mechanism so that movements can be timed and/or timestamped. GPS tracking would allow a user to monitor their distance moved which could be useful when the circuit is incorporated into a support bandage, for example by showing the optimal movement range to aid recovery.

A further relevant parameter that the circuit can be adapted to measure is the reflectance of a surface. If the circuit layer that is closest to a surface (i.e. one of the terminal circuit layers) incorporates one or more LEDs and light sensors, then the amount of light reflected from the surface can be determined. Using a series of LEDs and light sensors allows for a comparative analysis of the reflectance across a circuit. This could be useful when, for example, a user wishes to compare the reflectance of a healthy skin at the edge of a bandage or wound to the reflectance of some afflicted part of the skin.

A further improvement to the described circuit could be implemented by incorporating piezoelectric elements into the system. The sensitivity of such elements to small disturbances makes them suited for the detection of vibrations (including sounds). Having a plurality of vibration sensing piezoelectric elements disposed at the nodes of the circuit and monitoring the magnitude of the vibrations detected at each can allow the source of vibrations to determined. When the circuit is employed in, for example, clothing, bandages or wound dressings, the origin points of internal vibrations can be determined. Acoustic monitoring of the body can inform a user of the health of the person to which the circuit is affixed through information pertaining to their bones, bowels or the heart through the use of a ballistocardiography.

Such piezoelectric elements could also be used to cause the circuit to exert its own force on an object. For example, where the circuit is incorporated into a support bandage, a current could be supplied to series of piezoelectric elements in order to apply pressure and stimulate blood flow in order to prevent deep-vein thrombosis. The amount of supplied current may be controlled by the processor. Piezoelectric elements may be used to provide energy to the circuit by harvesting the energy of distortions in the circuit; i.e. by harvesting applied to mechanically distort the circuit.

The circuit may also be adapted to communicate wirelessly via a communication interface using for example, radio, Bluetooth®, WiFi® or Near-field Communication (NFC). This may be achieved by incorporating conventional antennae into the circuitry or the nodes may be dimensioned appropriately so as to be able to receive the relevant frequencies, for example several nodes may form a monopole or dipole antenna. Such functionality would allow the circuit to communicate with another device, such as a PC, laptop, smartphone, smartwatch, tablet or other flexible circuits.

The ability to communicate with external devices further increases the functionality of a circuit by allowing users or others to obtain information determined by the circuit or stored thereon. For example, it may be advantageous for measurements made by the circuit or system information such as power level to be relayed to another device with further capabilities, such as a user's smartphone which can perform further processing or simply display collected data. A smartphone, tablet, smartwatch or some other smart device may be used to coach a user by relaying information regarding, for example, the amount of energy exerted in movement necessary to achieve optimal recovery from an injury. This process could be gamified to further motivate and incentivise proper use. An RFID tag may be used to store and convey information about the circuit, such as a unique ID number when the circuit was installed, information about a patient on which the circuit is installed or the circuit's operational performance. The system could also be deployed as a component of a blockchain system, storing such information in a distributed database.

A further improvement to the circuit can be made by careful consideration of how its power is supplied. A conventional power supply such as an external wired supply or an internal battery (by which is meant any electrical storage device) may be used to supply power to the circuitry. Power may also be passively or actively supplied through the wireless methods described above by utilising the current generated in an antenna when receiving an electromagnetic wave. Such receivers could also be used to power the circuit by harvesting energy from ambient, incidental radio sources. Similarly, inductors can be used to passively harvest energy from ambient magnetic fields as the circuit moves therethrough. Another approach by which the circuit may produce its own power is through the provision of two adjacent circuit layers separated by an insulating layer acting as an electrochemical cell. In this arrangement, an electrode on one circuit layer acts as an anode and an electrode on the other circuit layer acts as a cathode. These electrodes may be composed of common corresponding electrode materials, such as a lithium or zinc anode paired with a carbon cathode. To implement this approach of power supply, the insulating layer (which by definition is insulating in its normal/unaltered state) must be able to facilitate ion transfer. To achieve this, the insulating layer may be imbued with an electrolyte, such as an electrolyte liquid or paste.

Particularly in devices where available power is limited, the careful control and management of power may be of importance. The circuit may manage its power resources by controlling which kind and how much data is to be used and transmitted in dependence on the criticality of that data. For example, when the circuit is in a low power state, only the most critical of data may be collected, analysed and transmitted and when the circuit is in a high power state any and all optional data may also be collected, analysed and transmitted. The circuit may automatically enter a low power, initiation state when it is first powered on (which could be automatic upon removal from packaging for example), in which it may not collect and analyse data that until it enters a normal operating mode. Such a phased power on sequence can be beneficial in a situation where data is not useful until the device is properly in place, for example, a wound dressing incorporating the circuit may not attempt to detect liquids until it has been in place for a certain amount of time.

In an example implementation, the circuit may initially be encased in packaging. The circuit may power on or initialise in response to the removal of packaging on one side of the circuit and the application of the removed packaging to the other side of the circuit. As well as initialising or powering on in response to the removal of packaging, the circuit may also initialise or power on when it is brought into contact (or close proximity) with a surface. This contact or proximity may be detected by the sensors discussed above (e.g. through measured capacitance or through measured resistance when a current passed through the surface), or it may be detected by a dedicated proximity sensor, for example an optical or infrared sensor or a radar system.

As already discussed in reference to wireless communication, it can be advantageous to have the circuit provide an indication and/or alert in response to some stimuli, for example, the detection of a liquid, liquid saturation, distortion, a pH change, expiration of a period of time (e.g. the shelf life or storage life of the circuit). Methods of indication include using a flexible electronic-ink (conventional or graphene-based e-ink or e-paper), a video monitor display, blinking lights/LEDs, vibrations mechanisms, speakers. A display may provide a visual indication of a desired movement pattern of the user, e.g. a desired movement pattern of one or more of the user's limbs. The desired movement pattern may be one to facilitate an optimal recovery. For example, the display may show a model of the skeleton and/or muscular system of a user and show current limb movements measured through the flexible circuit and display a comparison of this movement to the optimal, or desired movement pattern, thus encouraging the user to recover correctly. Alternatively, the display may provide a visual indication of a quantified amount of movement of one or more of the user's limbs over a specified period of time as measured by the circuit, and a comparison of this level of movement with a recommended value to promote recovery. That recommended value may be a dynamic value, i.e. it may change in time in accordance with a recovery protocol to reflect the recovery of the user over time. Reminders could be provided to a user to remind the user to undertake more physical exercise in order to recover quicker. A linear (one-dimensional) or matrix (two-dimensional) barcode could be provided on the circuit, with information pertaining to the identity of the circuit encoded thereon.

Several of the features described above may benefit from the inclusion of a method/mechanism for timing and/or time-stamping events. A clock signal may be generated by, for example, a crystal oscillator or a phase-locked loop.

A further improvement to the circuit, particularly when used as part of a wound dressing or bandage, is the inclusion of a releasable medicament. An openable, breakable or dissolvable capsule may be used to store the medicament. The medicament may be released on receiving an input from a user or in response to the expiry of a specified time period since the dressing/bandage was applied or activated or automatically in dependence on one or more of the measurable parameters discussed above (e.g. a distortion caused by swelling is detected or a change in pH or receiving a predetermined applied distortion). The capsule may break open in response to receiving the predetermined applied distortion. The capsule may comprise a frangible region (e.g. a perforated region or a thin-walled region) that is inclined to break upon receiving the predetermined applied distortion. The medicament may also be held in the insulating layers, for example by the medicament being absorbed into the insulating layers. The circuit layers may form a mesh through which the medicament may pass. As mentioned above, in reference to providing indications to measuring reflectance and to providing indications to a user, the circuit may incorporate one or more LEDs. These LEDs may be used to activate the medicament in the case where it is a light activated—e.g. a light-activated skin cream—by outputting light of a predetermined wavelength onto the medicament. Layers of graphene may be used to filter out specific wavelengths of light, allowing only predetermined wavelengths to penetrate the entire circuit.

The circuit layers may be an application-specific integrated circuit (ASIC) a programmable logic array, a field-programmable gate array (FPGA). The circuit layers may be graphene circuitry comprising a single graphene sheet, multiple individual sheets, or smaller graphene elements (such as nanoflakes) deposited on a substrate.

This deposition may be done onto a wide variety of substrates including fabrics, plastics and resins. The deposited substance should be immediately dried and then sandwiched between insulating layers.

The processor may be any kind of device, machine or dedicated circuit, or collection or portion thereof, with processing capability such that it can execute instructions. A processor may be any kind of general purpose or dedicated processor, such as a CPU, GPU, System-on-chip, state machine, media processor, an application-specific integrated circuit, a programmable logic array, a field-programmable gate array (FPGA), or the like. A computer or computer system may comprise one or more processors.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A flexible circuit comprising:
two or more circuit layers separated by one or more insulating layers;
a plurality of nodes located on each of the circuit layers; and
a processor configured to estimate the distribution of liquid within the circuit from the capacitance measured at one or more of the nodes.

2. A flexible circuit as claimed in claim 1, wherein the processor is further configured to, by comparing the capacitance with known values, at least one of: identify the liquid, determine the pH of the liquid, and determine the salinity of the liquid.

3. A flexible circuit as claimed in claim 1, wherein the processor is further configured to estimate the rate at which liquid passes through the circuit by comparing the distribution of liquid within the circuit two or more points in time.

4. A flexible circuit as claimed in claim 1, wherein the processor is further configured to estimate the distortion of the circuit from the capacitance measured at one or more of the nodes.

5. A flexible circuit as claimed in claim 1, wherein one or more of the circuit layers further comprise a strain gauge.

6. A flexible circuit as claimed in claim 4, wherein the processor is further configured to one or more of: increment a counter when the distortion exceeds a predetermined threshold, and continuously record the distortion of the circuit.

7. (canceled)

8. A flexible circuit as claimed claim 4, wherein the processor is configured to compensate for certain predefined distortions by ignoring the distortion detected by a subset of the nodes.

9. A flexible circuit as claimed claim 4, arranged such that, when affixed to a user and in use, the circuit layers are situated for detecting distortion caused by at least one of: cardiovascular activity, and respiration.

10. A flexible circuit as claimed in claim 9, wherein the processor is further configured to estimate the heart rate or the respiratory rate of a user based, at least in part, on the detected distortion caused by cardiovascular activity or respiration respectively.

11. A flexible circuit as claimed in claim 9, wherein the processor is further configured to receive data from another flexible circuit and, in dependence on the received data and the detected distortion caused by at least one of cardiovascular activity and respiration, to determine a pulse wave velocity.

12. A flexible circuit as claimed in claim 1, wherein one or more of the nodes are arranged such that, when the circuit is affixed to a user and in use, the nodes measure the electrical activity of the skin of the user underlying the circuit.

13. A flexible circuit as claimed in claim 12, comprising a common ground wire for connecting to another flexible circuit and wherein the processor is further configured to receive data from two connected flexible circuits and, in dependence the received data and the detected electrical activity of the skin of the user underlying the circuit, to determine an ECG reading using Einthoven's triangle effects.

14. A flexible circuit as claimed in claim 12, wherein the processor is further configured to estimate the heart rate of a user based, at least in part, on the detected electrical activity of the skin of the user underlying the circuit.

15.-25. (canceled)

26. A flexible circuit as claimed in claim 1, wherein the nodes are dimensioned so as to allow the reception and transmission of radio frequencies.

27.-29. (canceled)

30. A flexible circuit as claimed in claim 26, wherein the processor is configured to enter a low power state in which only data critical to the operation of the circuit is transmitted when the circuit is first powered on or in response to an input from a user, and enter a high power state, in which all data may be transmitted, when the circuit has been powered on for a predetermined length of time or in response to an input from a user.

31. A flexible circuit as claimed in claim 30, wherein the circuit is configured to not take measurements at the nodes when in the low power state.

32.-33. (canceled)

34. A flexible circuit as claimed in claim 1, further comprising an indicator configured to provide an indication of measured parameters to a user, the indicator comprising at least one of: a video display, an e-ink display, blinking light(s), LED(s), a vibration mechanism, a speaker.

35.-48. (canceled)

49. A flexible circuit as claimed in claim 1, wherein each circuit layer is connected to a respective processor and wherein measurements taken by the nodes on each circuit layer are relayed to their respective processors.

50. A flexible circuit as claimed in claim 1, wherein the flexible circuit is incorporated into at least one of: a wound dressing, a bandage, a support bandage, compression socks, and clothing.

51. A flexible circuit as claimed in claim 1, wherein the circuit layers comprise printed graphene circuitry.

52.-55. (canceled)
